(19) United States
(12) Patent Application Publication
(10) Pub. No.: US 2022/0412976 A1
(43) Pub. Date: Dec. 29, 2022

(54) CLINICAL MANAGEMENT OF OROPHARYNGEAL SQUAMOUS CELL CARCINOMA

(71) Applicant: AMLO Biosciences Limited, Newcastle Upon Tyne (GB)

(72) Inventor: Penny LOVAT, Newcastle Upon Tyne (GB)

(21) Appl. No.: 17/633,073

(22) PCT Filed: Jun. 25, 2020
(86) PCT No.: PCT/GB2020/051539

§ 371 Date: Feb. 4, 2022

(30) Foreign Application Priority Data
Aug. 6, 2019 (GB) 1911210.1

(51) Int. Cl. G01N 33/574; C07K 16/28; A61K 33/243; A61P 35/00; G01N 33/68

(52) U.S. Cl. CPC G01N 33/57407; C07K 16/2863; A61K 33/243; A61P 35/00; G01N 33/6863; C07K 2317/24; G01N 2800/52

(57) ABSTRACT

The present invention relates inter alia to methods of determining whether or not a subject suffering from oropharyngeal squamous cell carcinoma (OPSCC) is suitable for de-escalated treatment. The invention also provides methods of treating OPSCC, and associated assays and kits.

### FIELD OF THE INVENTION

The present invention relates inter alia to methods of determining whether or not a subject suffering from oropharyngeal squamous cell carcinoma (OPSCC) is suitable for de-escalated treatment. The invention also provides methods of treating OPSCC, and associated assays and kits.

### BACKGROUND TO THE INVENTION

OPSCC is a rapidly increasing malignancy, representing one quarter of total head and neck cancers (HNC) and causing around 97,000 deaths/year worldwide. Infection with human papillomavirus (HPV) represents a key factor in OPSCC development.

Although HPV promotes tumorigenesis, patients with HPV-positive OPSCC have a more favourable prognosis and response to treatment compared to their HPV-negative counterparts (Albers et al., 2017 Nature 7: 16715). This has stimulated recent debate over treatment de-escalation for HPV-positive OPSCC. However, within the HPV+ cohort there is a cohort that respond poorly to treatment. Thus, there remains an unmet need for credible biomarkers to identify genuinely low risk HPV-positive tumor subsets to support de-escalated treatment regimens.

It is an aim of some embodiments of the present invention to at least partially mitigate some of the problems identified in the prior art.

### SUMMARY OF CERTAIN EMBODIMENTS OF THE INVENTION

Certain aspects of the present invention relate to the unexpected finding that Ambra-1 can be used as a marker to identify low risk subsets of HPV positive OPSCC suitable for de-escalated treatment. Less intensive treatment may achieve similar efficacy in low-risk subjects with less toxicity and an improved quality of life. Conversely, Ambra-1 can be used to identify high risk subsets of HPV positive OPSCC that are not suitable for de-escalated treatment. More intensive treatment (e.g. adjuvant therapy) may be administered to high-risk subjects.

In particular, the inventors have surprisingly shown that loss of Ambra-1 protein in HPV positive tumors is indicative of a tumor that is low risk (such that treatment may be de-escalated). Conversely, retention of Ambra-1 protein in HPV positive tumors is indicative of a tumor that is high risk (such that treatment may not be de-escalated).

Accordingly, the invention provides:
a method of determining whether or not a subject suffering from HPV positive OPSCC is suitable for de-escalated treatment,
the method comprising determining the expression of Ambra-1 in an OPSCC tissue sample obtained from the subject, wherein:
(i) retained or increased expression of Ambra-1 indicates the subject is not suitable for de-escalated treatment; or
(ii) decreased or loss in expression of Ambra-1 indicates the subject is suitable for de-escalated treatment.
a method of treating a subject suffering from HPV positive OPSCC comprising:
(i) determining whether or not the subject is suitable for de-escalated treatment according to any method described herein; and
(ii) (a) if the subject is not suitable for de-escalated treatment, administering an adjuvant therapy to the subject; or
   (b) if the subject is suitable for de-escalated treatment, administering a de-escalated treatment to the subject.
an in vitro assay for determining whether or not a subject suffering from HPV positive OPSCC is suitable for de-escalated treatment, the assay comprising:
contacting OPSCC tissue in a sample obtained from the subject with a ligand specific for Ambra-1 as described herein, wherein the presence of Ambra-1 creates an Ambra-1-ligand complex; and
detecting and/or quantifying the Ambra-1-ligand complex.
a kit for determining whether or not a subject suffering from HPV positive oropharyngeal OPSCC is suitable for de-escalated treatment, the kit comprising a ligand specific for Ambra-1 as described herein.

### DETAILED DESCRIPTION OF CERTAIN EMBODIMENTS

Embodiments of the invention will now be described by way of example only, and with reference to the accompanying Figures which show the relationship between HPV status and Ambra-1 expression with overall survival in the Newcastle Discovery OPSCC cohort.

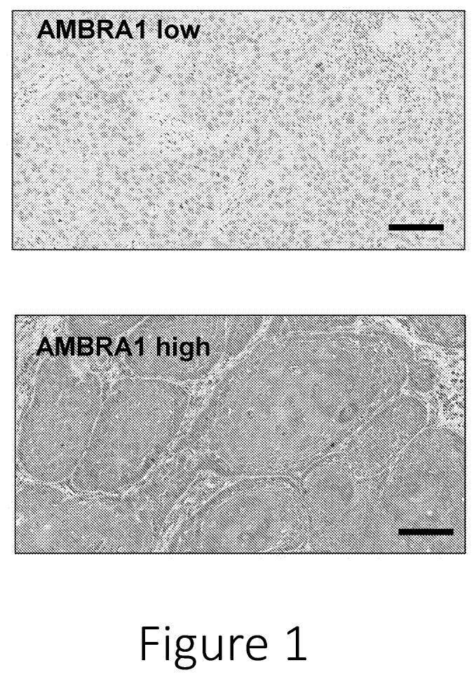

FIG. 1 shows a Representative photomicrograph of “low” (h score<140) and “high” (h score= or >140) immunohistochemical Ambra-1 staining in OPSCC TMA (scale bars=100 μm). Overall survival rates were determined by Kaplan-Meier analysis and compared by Log-Rank test.

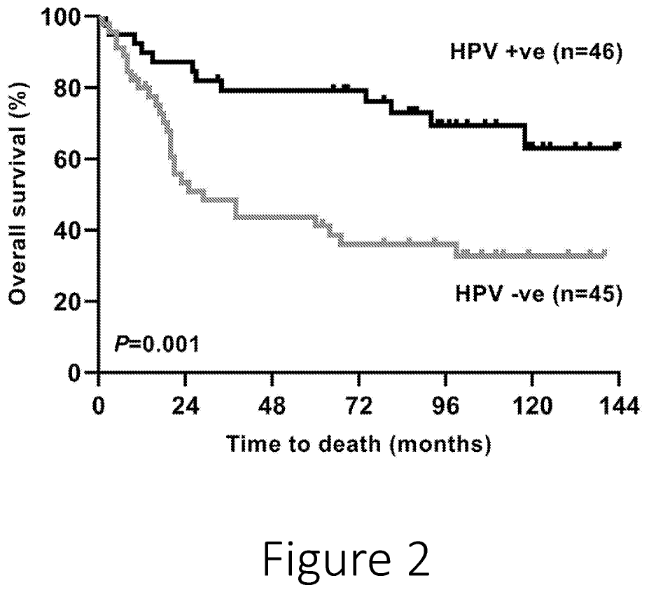

FIG. 2 shows HPV positive versus HPV negative OPSCC samples (OS=63% versus 32.7%; P=0.0012 HR 2.87 (95% CI 1.54-5.35).

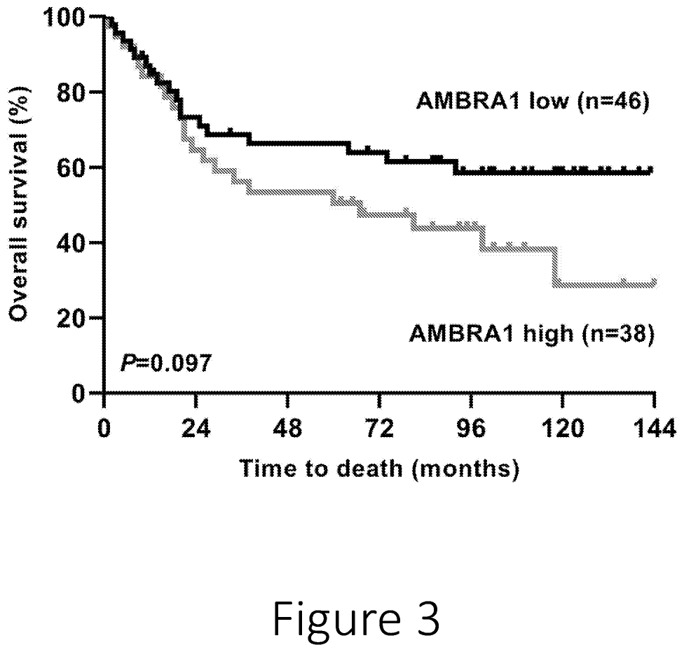

FIG. 3 shows Ambra-1 “low” versus Ambra-1 “high” (OS=58.5% versus 28.7%; P=0.0972 HR 1.68 (95% CI 0.90-3.14).

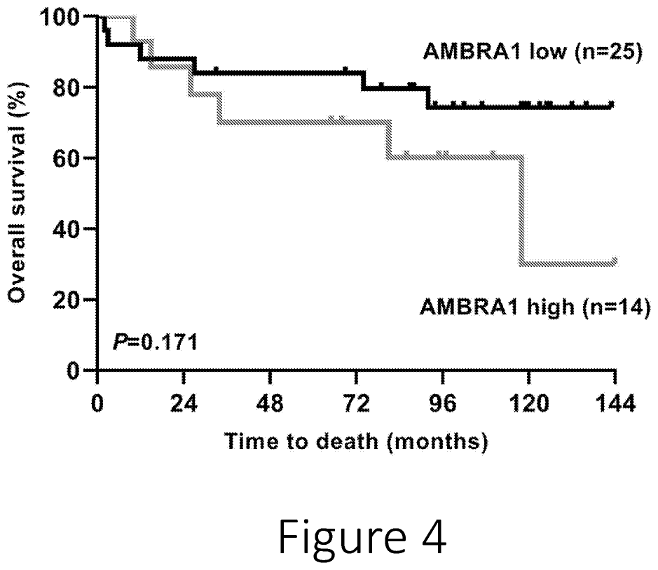

FIG. 4 shows Ambra-1 “low” versus AMBRA1 “high” within the HPV positive sub-group only (OS=74.3% versus 30.1%; P=0.1712 HR 2.15 (95% CI 0.67-7.25).

### SEQUENCE LISTING

SEQ ID NO: 1 shows the amino acid sequence of HCDR1 of the anti-Ambra-1 antibody (SYWIH);

SEQ ID NO: 2 shows the amino acid sequence of HCDR2 of the anti-Ambra-1 antibody

(TIFPSRSYTTYSPSFQG);

SEQ ID NO: 3 shows the amino acid sequence of HCDR3 (DTPSTALKSPFDY) of the anti-Ambra-1 antibody;

SEQ ID NO: 4 shows the amino acid sequence of LCDR1 of the anti-Ambra-1 antibody

(SGSSSNIGYNYVY);

SEQ ID NO: 5 shows the amino acid sequence of LCDR2 of the anti-Ambra-1 antibody

(ENNKRPS);

SEQ ID NO: 6 shows the amino acid sequence of LCDR3 of the anti-Ambra-1 antibody

(SSWDSHSNSYV);

SEQ ID NO: 7 shows the amino acid sequence of HCFR1 of the anti-Ambra-1 antibody

(EVQLVQSGAEVKKPGESLKISCKGSGYSFS);

SEQ ID NO: 8 shows the amino acid sequence of HCFR2 of the anti-Ambra-1 antibody

(WVRQMPGKGLEWMG);

SEQ ID NO: 9 shows the amino acid sequence of HCFR3 of the anti-Ambra-1 antibody

(QVTISADKSISTAYLQWSSLKASDTAMYYCAR);

SEQ ID NO: 10 shows the amino acid sequence of HCFR4 of the anti-Ambra-1 antibody

(WGQGTLVTVSS);

SEQ ID NO: 11 shows the amino acid sequence of LCFR1 of the anti-Ambra-1 antibody

(DIVLTQPPSVSGAPGQRVTISC

SEQ ID NO: 12 shows the amino acid sequence of LCFR2 of the anti-Ambra-1 antibody

(WYQQLPGTAPKLLIY);

SEQ ID NO: 13 shows the amino acid sequence of LCFR3 of the anti-Ambra-1 antibody

(GVPDRFSGSKSGTSASLAITGLQAEDEADYYC);

SEQ ID NO: 14 shows the amino acid sequence of LCFR4 of the anti-Ambra-1 antibody

(FGGGTKLTVLGQ);

SEQ ID NO: 15 shows the amino acid sequence of the V_{H} domain of the anti-Ambra-1 antibody:

EVQLVQSGAEVKKPGESLKISCKGSGYSFSSYWIHWVRQMP
GKGLEWMGTIFPSRSYTTYSPSFQGQVTISADKSISTAYLQ
WSSLKASDTAMYYCARDTPSTALKSPFDYWGQGTLVTVSS

SEQ ID NO: 16 shows the amino acid sequence of the V_{L} domain of the anti-Ambra-1 antibody;

DIVLTQPPSVSGAPGQRVTISCSGSSSNIGYNYVYWYQQL
PGTAPKLLIYENNKRPSGVPDRFSGSKSGTSASLAITGLQ
AEDEADYYCSSWDSHSNSYVFGGGTKLTVLGQ

SEQ ID NO: 17 shows the amino acid sequence of the Fd chain (V_{H} domain and constant domain) of the Fab region of the anti-Ambra-1 antibody;

EVQLVQSGAEVKKPGESLKISCKGSGYSFSSYWIHWVRQM
PGKGLEWMGTIFPSRSYTTYSPSFQGQVTISADKSISTAY
LQWSSLKASDTAMYYCARDTPSTALKSPFDYWGQGTLVTV
SSASTKGPSVFPLAPSSKSTSGGTAALGCLVKDYFPEPVT
VSWNSGALTSGVHTFPAVLQSSGLYSLSSVVTVPSSSLGT
QTYICNVNHKPSNTKVDKKVEPKS

SEQ ID NO: 18 shows the amino acid sequence of the light chain (V_{L} domain and constant domain) of the Fab region of the anti-Ambra-1 antibody;

DIVLTQPPSVSGAPGQRVTISCSGSSSNIGYNYVYWYQQLPGTA
PKLLIYENNKRPSGVPDRFSGSKSGTSASLAITGLQAEDEADYY
CSSWDSHSNSYVFGGGTKLTVLGQPKAAPSVTLFPPSSEELQAN
KATLVCLISDFYPGAVTVAWKADSSPVKAGVETTTPSKQSNNKY
AASSYLSLTPEQWKSHRSYSCQVTHEGSTVEKTVAPTEA

SEQ ID NO: 19 shows the nucleic acid sequence encoding the Fd chain of the Fab region and tags (alkaline phosphatase dimerization domain sequence (AP), FLAG tag (DYKDDDDK) and His6 tag of the anti-Ambra-1 antibody;

GAAGTGCAATTGGTGCAGAGCGGTGCGGAAGTGAAAAAAC
CGGGCGAAAGCCTGAAAATTAGCTGCAAAGGCTCCGGATA
TAGCTTCTCTTCTTACTGGATCCATTGGGTGCGCCAGATG
CCGGGCAAAGGTCTCGAGTGGATGGGCACTATCTTCCCGT
CTCGTAGCTACACCACTTATAGCCCGAGCTTTCAGGGCCA
GGTGACCATTAGCGCGGATAAAAGCATCAGCACCGCGTAT
CTGCAATGGAGCAGCCTGAAAGCGAGCGATACCGCGATGT
ATTATTGCGCGCGTGACACTCCGTCTACTGCTCTGAAATC
TCCGTTCGATTACTGGGGCCAAGGCACCCTGGTGACTGTT
AGCTCAGCGTCGACCAAAGGCCCGAGCGTGTTTCCGCTGG
CCCCGAGCAGCAAAAGCACCAGCGGCGGCACCGCCGCACT
GGGCTGCCTGGTGAAAGATTATTTCCCGGAACCAGTGACC
GTGAGCTGGAACAGCGGTGCCCTGACCAGCGGCGTGCATA
CCTTTCCGGCGGTGCTGCAAAGCAGCGGCCTGTATAGCCT
GAGCAGCGTTGTGACCGTGCCGAGCAGCAGCCTGGGCACC
CAGACCTATATTTGCAACGTCAACCATAAACCGAGCAACA
CCAAAGTCGATAAAAAAGTCGAACCGAAAAGCGAATTCAA
GGCTGAAATGCCTGTTCTGGAAAACCGGGCTGCTCAGGGC
GATATTACTACACCCGGCGGTGCTCGCCGTTTAACGGGTG
ATCAGACTGCCGCTCTGCGTGATTCTCTTAGCGATAAACC
TGCAAAAAATATTATTTTGCTGATTGGCGATGGGATGGGG
GACTCGGAAATTACTGCCGCACGTAATTATGCCGAAGGTG
CGGGCGGCTTTTTTAAAGGTATAGATGCCTTACCGCTTAC
CGGGCAATACACTCACTATGCGCTGAATAGAAAAACCGGC
AAACCGGACTACGTCACCAGCTCGGCTGCATCAGCAACCG
CCTGGTCAACCGGTGTCAAAACCTATAACGGCGCGCTGGG
CGTCGATATTCACGAAAAAGATCACCCAACGATTCTGGAA
ATGGCAAAAGCCGCAGGTCTGGCGACCGGTAACGTTTCTA
CCGCAGAGTTGCAGGATGCCACGCCCGCTGCGCTGGTGGC
ACATGTGACCTCGCGCAAATGCTACGGTCCGAGCGCGACC
AGTGAAAAATGTCCGGGTAACGCTCTGGAAAAAGGCGGAA
AAGGATCGATTACCGAACAGCTGCTTAACGCTCGTGCCGA
CGTTACGCTTGGCGGCGGCGCAAAAACCTTTGCTGAAACG
GCAACCGCTGGTGAATGGCAGGGAAAAACGCTGCGTGAAC
AGGCACAGGCGCGTGGTTATCAGTTGGTGAGCGATGCTGC
CTCACTGAACTCGGTGACGGAAGCGAATCAGCAAAAACCC
CTGCTTGGCCTGTTTGCTGACGGCAATATGCCAGTGCGCT
GGCTAGGACCGAAAGCAACGTACCATGGCAATATCGATAA
GCCCGCAGTCACCTGTACGCCAAATCCGCAACGTAATGAC
AGTGTACCAACCCTGGCGCAGATGACCGACAAAGCCATTG
AATTGTTGAGTAAAAATGAGAAAGGCTTTTTCCTGCAAGT
TGAAGGTGCGTCAATCGATAAACAGGATCATGCTGCGAAT
CCTTGTGGGCAAATTGGCGAGACGGTCGATCTCGATGAAG
CCGTACAACGGGCGCTGGAGTTCGCTAAAAAGGAGGGTAA
CACGCTGGTCATAGTCACCGCTGATCACGCCCACGCCAGC
CAGATTGTTGCGCCGGATACCAAAGCTCCGGGCCTCACCC
AGGCGCTAAATACCAAAGATGGCGCAGTGATGGTGATGAG
TTACGGGAACTCCGAAGAGGATTCACAAGAACATACCGGC
AGTCAGTTGCGTATTGCGGCGTATGGCCCGCATGCCGCCA
ATGTTGTTGGACTGACCGACCAGACCGATCTCTTCTACAC
CATGAAAGCCGCTCTGGGGCTGAAAGGCGCGCCGGACTAT
AAAGATGACGATGACAAAGGCGCGCCGCACCATCATCAC
CATCAC

SEQ ID NO: 20 shows the nucleic acid sequence encoding the light chain of the Fab region of the anti-Ambra-1 antibody;

GATATCGTGCTGACCCAGCCGCCGAGCGTGAGCGGTGCAC
CGGGCCAGCGCGTGACCATTAGCTGTAGCGGCAGCAGCAG
CAACATTGGTTACAACTACGTGTACTGGTACCAGCAGCTG
CCGGGCACGGCGCCGAAACTGCTGATCTACGAAAACAACA
AACGCCCGAGCGGCGTGCCGGATCGCTTTAGCGGATCCAA
AAGCGGCACCAGCGCCAGCCTGGCGATTACCGGCCTGCAA
GCAGAAGACGAAGCGGATTATTACTGCTCTTCTTGGGACT
CTCATTCTAACTCTTACGTGTTTGGCGGCGGCACGAAGTT
AACCGTTCTTGGCCAGCCGAAAGCCGCCCCAAGCGTGACC
CTGTTTCCGCCGAGCAGCGAAGAACTGCAAGCCAACAAAG
CCACCCTGGTTTGCCTGATCAGCGATTTTTATCCGGGTGC
CGTGACCGTGGCCTGGAAAGCCGATAGCAGCCCGGTGAAA
GCCGGCGTGGAAACCACCACCCCGAGCAAACAGAGCAACA
ACAAATATGCCGCCAGCAGCTATCTGAGCCTGACCCCGGA
ACAGTGGAAAAGCCATCGCAGCTATAGTTGTCAAGTGACC
CATGAAGGCAGCACCGTGGAAAAAACCGTGGCCCCGACCG
AGGCC

SEQ ID NO: 21 shows the amino acid sequence of human Ambra-1;

MKVVPEKNAVRILWGRERGARAMGAQRLLQELVEDKTRWM
KWEGKRVELPDSPRSTFLLAFSPDRTLLASTHVNHNIYIT
EVKTGKCVHSLIGHRRTPWCVTFHPTISGLIASGCLDGEV
RIWDLHGGSESWFTDSNNAIASLAFHPTAQLLLIATANEI
HFWDWSRREPFAVVKTASEMERVRLVRFDPLGHYLLTAIV
NPSNQQGDDEPEIPIDGTELSHYRQRALLQSQPVRRTPLL
HNFLHMLSSRSSGIQVGEQSTVQDSATPSPPPPPPQPSTE
RPRTSAYIRLRQRVSYPTAECCQHLGILCLCSRCSGTRVP
SLLPHQDSVPPASARATTPSFSFVQTEPFHPPEQASSTQQ
DQGLLNRPSAFSTVQSSTAGNTLRNLSLGPTRRSLGGPLS
SHPSRYHREIAPGLTGSEWTRTVLSLNSRSEAESMPPPRT
SASSVSLLSVLRQQEGGSQASVYTSATEGRGFPASGLATE
SDGGNGSSQNNSGSIRHELQCDLRRFFLEYDRLQELDQSL
SGEAPQTQQAQEMLNNNIESERPGPSHQPTPHSSENNSNL
SRGHLNRCRACHNLLTFNNDTLRWERTTPNYSSGEASSSW
QVPSSFESVPSSGSQLPPLERTEGQTPSSSRLELSSSASP
QEERTVGVAFNQETGHWERIYTQSSRSGTVSQEALHQDMP
EESSEEDSLRRRLLESSLISLSRYDGAGSREHPIYPDPAR
LSPAAYYAQRMIQYLSRRDSIRQRSMRYQQNRLRSSTSSS
SSDNQGPSVEGTDLEFEDFEDNGDRSRHRAPRNARMSAPS
LGRFVPRRFLLPEYLPYAGIFHERGQPGLATHSSVNRVLA
GAVIGDGQSAVASNIANTTYRLQWWDFTKFDLPEISNASV
NVLVQNCKIYNDASCDISADGQLLAAFIPSSQRGFPDEGI
LAVYSLAPHNLGEMLYTKRFGPNAISVSLSPMGRYVMVGL
ASRRILLHPSTEHMVAQVFRLQQAHGGETSMRRVFNVLYP
MPADQRRHVSINSARWLPEPGLGLAYGTNKGDLVICRPEA
LNSGVEYYWDQLNETVFTVHSNSRSSERPGTSRATWRTDR
DMGLMNAIGLQPRNPATSVTSQGTQTLALQLQNAETQTER
EVPEPGTAASGPGEGEGSEYGASGEDALSRIQRLMAEGGM
TAVVQREQSTTMASMGGFGNNIIVSHRIHRSSQTGTEPGA
AHTSSPQPSTSRGLLPEAGQLAERGLSPRTASWDQPGTPG
REPTQPTLPSSSPVPIPVSLPSAEGPTLHCELTNNNHLLD
GGSSRGDAAGPRGEPRNR

(DYKDDDDK)

SEQ ID NO:22 shows the amino acid sequence of an exemplary epitope Lag

SEQ ID NO: 23 shows the amino acid sequence of an exemplary epitope tag

(GKPIPNPLLGLDST)

(WSHPQFEK)

SEQ ID NO: 24 shows the amino acid sequence of an exemplary epitope tag

(HHHHHH)

SEQ ID NO: 25 shows the amino acid sequence of the His6 tag

SEQ ID NO: 26 shows the amino acid sequence of the peptide to which anti-Ambra-1 antibodies were raised

(CGGSSRGDAAGPRGEPRNR)

SEQ ID NO: 27 shows the Ambra-1 C-terminal sequence used for replacement analysis in epitope mapping

(VSLPSAEGPTLHCELTNNNHLLDGGSSRGDAAGPRGEPRNR)

SEQ ID NO: 28 shows the REPNET epitope of the Anti-Ambra-1 antibodies

(DGGSSRGDAAGPRGEPRNR)

(HLLDGGSSR)

SEQ ID NO: 29 shows the LIN epitope of the Anti-Ambra-1 antibodies

(NHLLDGGSSR)

SEQ ID NO: 30 shows the LOOP epitope of the Anti-Ambra-1 antibodies

(EPRN)

SEQ ID NO: 31 shows a core epitope of the Anti-Ambra-1 antibodies

The practice of embodiments of the present invention employs, unless otherwise indicated, conventional techniques of chemistry, molecular biology, pharmaceutical formulation, pharmacology and medicine, which are within the skill of those working in the art.

Most general chemistry techniques can be found in Comprehensive Heterocyclic Chemistry IF (Katritzky et al., 1996, published by Pergamon Press); Comprehensive Organic Functional Group Transformations (Katritzky et al., 1995, published by Pergamon Press); Comprehensive Organic Synthesis (Trost et al, 1991, published by Pergamon); Heterocyclic Chemistry (Joule et al. published by Chapman & Hall); Protective Groups in Organic Synthesis (Greene et al., 1999, published by Wiley-Interscience); and Protecting Groups (Kocienski et al., 1994).

Most general molecular biology techniques can be found in Sambrook et al, Molecular Cloning, A Laboratory Manual (2001) Cold Harbor-Laboratory Press, Cold Spring Harbor, N.Y. or Ausubel et al., Current Protocols in Molecular Biology (1990) published by John Wiley and Sons, N.Y.

Most general pharmaceutical formulation techniques can be found in Pharmaceutical Preformulation and Formulation (2^{nd} Edition edited by Mark Gibson) and Pharmaceutical Excipients: Properties, Functionality and Applications in Research and Industry (edited by Otilia M Y Koo, published by Wiley).

Most general pharmacological techniques can be found in A Textbook of Clinical Pharmacology and Therapeutics (5^{th} Edition published by Arnold Hodder).

Most general techniques on the prescribing, dispensing and administering of medicines can be found in the British National Formulary 72 (published jointly by BMJ Publishing Group Ltd and Royal Pharmaceutical Society).

Unless defined otherwise, all technical and scientific terms used herein have the same meaning as commonly understood by one of ordinary skill in the art to which this disclosure belongs. For example, the Concise Dictionary of Biomedicine and Molecular Biology, Juo, Pei-Show, 2^{nd} ed., 2002, CRC Press; The Dictionary of Cell and Molecular Biology, 3^{rd} ed., Academic Press; and the Oxford University Press, provide a person skilled in the art with a general dictionary of many of the terms used in this disclosure. For chemical terms, the skilled person may refer to the International Union of Pure and Applied Chemistry (IUPAC).

Units, prefixes and symbols are denoted in their Système International d'Unités (SI) accepted form. Numeric ranges are inclusive of the numbers defining the range.

Treatment Regime for a Subject Suffering from OPSCC

The invention relates to methods of determining a treatment regime for a subject suffering from OPSCC. In certain embodiments, the methods comprise determining whether or not the subject is suitable for a de-escalated treatment. Typically, the methods are in vitro.

As used herein, the term “OPSCC” refers to a form of cancer in which malignant cells form in squamous cells of the oropharynx.

A subject who is “suitable for de-escalated treatment” typically means a subject who is at low risk (e.g. more likely to survive the carcinoma). For such subjects, a de-escalated treatment may be administered to reduce or avoid any adverse side-effects associated with adjuvant therapy.

A subject who is “not suitable for de-escalated treatment” typically means a subject who is at high risk (e.g. less likely to survive the carcinoma). For such subjects, a de-escalated treatment may not be administered. Instead, adjuvant therapy may be administered despite the risk of adverse side-effects.

Adverse-side effects associated with adjuvant therapy may include xerostomia, swallowing disorder, pain and stiffness of neck and/or ototoxicity.

The methods of the invention allow evaluation of potential risk based on evaluation of Ambra-1 expression in OPSCC tissue obtained from an individual subject. This may allow determination of whether or not to proceed and treat the subject with a de-escalated treatment or adjuvant therapy.

The subject may be a human or an animal suffering from OPSCC. In some embodiments, the subject is a horse, cat or dog. Typically, the subject is a human. Typically, the subject has already been diagnosed as having OPSCC. Typically, the subject is an individual for whom a de-escalated treatment or adjuvant therapy is being considered.

Typically, the subject is HPV positive. In some embodiments, the methods of the invention further comprises determining whether or not the subject is HPV positive or negative. Typically, the subject has already been diagnosed as HPV positive.

HPV diagnosis may rely on cytologic, histologic or molecular methods including DNA, RNA or protein-based tests. For example, p16 expression may be assessed by immunohistochemistry (IHC) to identify a subject as HPV positive. HPV DNA may be determined in tumor cell nuclei by in situ hybridization (ISH). HPV E6 and E7 mRNA (HPV oncogenes) may be detected e.g. by reverse transcriptase polymerase chain reaction (RT-PCR). Typically, marker expression is determined in fresh frozen tissue. A wide range of HPV tests for OPSCC are commercially available. See, for example, Kim et al. J Pathol Clin Res. 2018; (4) 213-226.

De-Escalated Treatment

As used herein, the term “de-escalated treatment” refers to a less intense, less aggressive and/or less toxic treatment regime as compared to a normal recognized care pathway.

A “normal recognized care pathway” may refer to a standard of care protocol. For example, a normal recognized care pathway may comprise post-operative high dose radiation and chemotherapy as described herein.

In certain embodiments, a normal recognized care pathway comprises concurrent radiotherapy and cisplatin-based chemotherapy following surgery. For example, the radiotherapy may comprise IMRT up to a dosage of about 70Gy. The chemotherapy may comprise up to about 200, about 250 or about 300 mg/m² cisplatin.

In certain embodiment, the de-escalated treatment comprises a reduced dosage of radiotherapy (e.g. IMRT). For example, a reduced dosage of radiotherapy (e.g. IMRT) may comprise up to about 65, up to about 60, up to about 55 or up to about 50Gy.

In certain embodiments, the de-escalated treatment comprises a reduced dosage of chemotherapy (e.g. cisplatin). For example, a reduced dosage may comprise up to about 100, about 125 or about 150 mg/m² cisplatin.

In certain embodiments, the de-escalated treatment avoids adjuvant therapy such as chemotherapy.

In certain embodiments, the de-escalated treatment avoids cisplatin-based chemotherapy.

In certain embodiments, the de-escalated treatment comprises less invasive surgery as compared to standard open surgery. For example, transoral laser microsurgery or transoral robotic surgery (TORS) may be used.

In certain embodiments, the de-escalated treatment comprises replacement of IMRT with proton beam therapy.

In certain embodiments, the de-escalated treatment comprises administration of a therapeutic antibody. For example, administration of chemotherapy (e.g. cisplatin) may be replaced with administration of a therapeutic antibody.

In certain embodiments, the therapeutic antibody is an epidermal growth factor receptor (EFGR) inhibitor (e.g. afatinib, lapatinib, dacomitinib or cetuximab). Typically, the therapeutic antibody is cetuximab.

In certain embodiments, the therapeutic antibody is a phosphoinositide 3-kinase pathway (P13K) inhibitor or checkpoint inhibitor (e.g. anti-PD-1 or anti-PD-L1).

The de-escalated treatment that is chosen may depend on the stage of the OPSCC tumor. The stage of the OPSCC is a description of how widespread it is. This includes its thickness in the skin, whether it has spread, and certain other factors. The stage is based on the results of the physical exam, biopsies, and any imaging tests (CT or MRI scan, etc.) or other tests that have been conducted. Such tests will be known to those skilled in the art. The system most often used to stage OPSCC is the American Joint Commission on Cancer (AJCC) TNM system. The Tables below describes the features identifying each stage for HPV negative and HPV positive OPSCC (AJCC, 8^{th} edition):

| | HPV negative OPSCC |
|---|---|
| Stage | (e.g. p16 negative) |
| TX | Primary tumor cannot be assessed |
| Tis | Carcinoma in situ |
| T1 | Tumor ≤2 cm in greatest dimension |
| T2 | Tumor >2 cm but not >4 cm greatest dimension |
| T3 | Tumor > than 4 cm in greatest dimension or extension to |
| | lingual surface of epiglottis |
| T4 | Moderately advanced or very advanced local disease |
| T4a | Moderately advanced local disease; tumor invades the |
| | larynx, extrinsic muscle of tongue, medial pterygoid, hard |
| | palate, or mandible |
| T4b | Very advanced local disease; tumor invades lateral |
| | pterygoid muscle, pterygoid plates, lateral nasopharynx, or |
| | skull base or encases carotid artery |

| | HPV positive OPSCC |
|---|---|
| Stage | (e.g. p16 positive) |
| T0 | No primary identified |
| T1 | Tumor ≤2 cm in greatest dimension |
| T2 | Tumor >2 cm but not >4 cm greatest dimension |
| T3 | Tumor > than 4 cm in greatest dimension or extension to |
| | lingual surface of epiglottis |
| T4 | Moderately advanced local disease; tumor invades the |
| | larynx, extrinsic muscle of tongue, medial pterygoid, hard |
| | palate, or mandible or beyond |

In certain embodiments, the methods of the invention may further comprise staging OPSCC in accordance with AJCC staging. Typically, the OPSCC has already been staged in accordance with AJCC staging.

In certain embodiments, the subject is suffering from HPV negative OPSCC and is stage TX, Tis, T1, T2, T3, T4, T4a or T4b.

In certain embodiments, the subject is suffering from HPV positive OPSCC and is stage T0, T1, T2, T3 or T4. Typically, the subject is early stage (e.g. T1 or T2) or late stage (e.g. T3 or T4). Typically, the subject is suffering from HPV positive OPSCC and is stage T1, T2, T3 or T4.

Primary Treatment

In certain embodiments, the subject is undergoing (or has undertaken) a primary treatment.

In certain embodiments, the primary treatment comprises surgery. Typically, surgery is performed to remove the primary carcinoma. Typically, the surgery is organ-sparing e.g. is intended to reduce functional morbidity or complications associated with large, invasive, open surgical approaches.

In certain embodiments, the surgery comprises robotic surgery. For example, transoral robotic surgery (TORS) may be used.

In certain embodiments, the primary treatment comprises radiotherapy. Typically, primary treatment includes both surgery and post-operative radiotherapy.

Any suitable radiotherapy may be used at any suitable dose for the subject.

In certain embodiments, the radiotherapy may comprise intensity-modulated radiation therapy (IMRT). For example, IMRT may be applied up to a dosage of about 70Gy. Typically, IMRT may be delivered in about 35 fractions over 6 weeks at six fractions per week (e.g. with two fractions given on one day, at least 6 hours apart). More typically, a subject may undergo IMRT once daily on days 1 to 4 and twice daily on day 5 weekly for 6 weeks.

The primary treatment that is chosen for the subject may depend on the stage of the OPSCC tumor. Typically, the subject is suffering from HPV positive OPSCC and is early stage (e.g. T1 or T2) or late stage (e.g. T3 or T4).

Adjuvant Treatment

In certain embodiments, a subject who is not suitable for de-escalated treatment is suitable for adjuvant therapy.

As used herein, the term “adjuvant therapy” refers to an additional therapy before, during or after the primary treatment. For example, an adjuvant therapy may comprise chemotherapy before, during or after post-operative high dose radiation as described herein.

Typically, the adjuvant therapy is toxic and/or may lead to adverse side-effects in the subject. As such, administration of the adjuvant therapy is preferably avoided in low-risk subsets of HPV positive OPSCC.

In certain embodiments, the adjuvant therapy is induction chemotherapy. For example, the induction chemotherapy may be docetaxel, cisplatin and/or 5-fluorouracil.

In certain embodiments, the adjuvant therapy is carboplatin and/or 5-fluorouracil.

In certain embodiments, the adjuvant therapy is chemotherapy. For example, the chemotherapy may be a platinum derivative, e.g. cisplatin, carboplatin and/or oxaliplatin.

In certain embodiments, the chemotherapy is cisplatin-based. For example, cisplatin may be administered concurrently with radiotherapy (e.g. IMPRT) as described herein. Typically, cisplatin is administered intravenously (IV). In certain embodiments, cisplatin is administered at a high dose, e.g. up to about 200, about 250 or about 300 mg/m². In certain embodiments, cisplatin is administered once or more during radiotherapy treatment (e.g. about 100 mg/m² on days 1 and 22 of IMRT radiotherapy, totaling about 200 mg/m²).

The type of adjuvant therapy may depend on the stage of the OPSCC tumor. Typically, the subject is suffering from HPV positive OPSCC and is early stage (e.g. T1 or T2) or late stage (e.g. T3 or T4).

Determining Expression of Ambra-1

In certain embodiments, the methods of the invention comprise determining the expression of Ambra-1 in an OPSCC tissue sample obtained from the subject. Typically, the tissue sample comprises at least a portion of OPSCC tissue and/or cells and expression of Ambra-1 protein is determined in the OPSCC tissues and/or cells.

In certain embodiments, the OPSCC tissue sample also comprises non-carcinoma tissue, e.g. normal tissue adjacent the OPSCC.

In certain embodiments, the tissue sample has previously been obtained from the subject such that the sampling itself does not form a part of the methods of the invention. The sample may have been obtained immediately prior to the method, or hours, days or weeks prior to the method. In other embodiments, a method of the invention may additionally comprise the step of obtaining the OPSCC tissue sample from the subject.

Typically, the OPSCC tissue sample may be a biopsy, or a section thereof, obtained from the subject. An OPSCC tissue sample, such as a biopsy, can be obtained through a variety of sampling methods known to those skilled in the art, including a punch biopsy, shave biopsy, wide local excision and other means. Aptly, the OPSCC tissue sample is taken from a surgical site from which the OPSCC has been excised from the subject.

Typically, the OPSCC tissue sample may be frozen, fresh, fixed (e.g. formalin fixed), centrifuged, and/or embedded (e.g. paraffin embedded), etc. The OPSCC tissue sample may be or have been subjected to a variety of well-known post-collection preparative and storage techniques (e.g., nucleic acid and/or protein extraction, fixation, storage, freezing, ultrafiltration, concentration, evaporation, centrifugation, etc.) prior to assessing the amount of Ambra-1 in the sample. Likewise, biopsies may also be subjected to post-collection preparative and storage techniques, e.g., fixation. An OPSCC tissue sample, or a section thereof, may be mounted on a solid support, such as a slide.

Ambra-1 (activating molecule in Beclin-1 regulated autophagy protein 1) is a WD40-containing protein. Studies have implicated Ambra-1 in the control of autophagy and cellular differentiation. The full length human amino acid sequence of Ambra-1 is set forth in SEQ ID NO: 21.

Typically, determining the expression of Ambra-1 in the OPSCC tissue sample comprises measuring the levels of Ambra-1 protein that may be present in the OPSCC tissue. This may be achieved by methods known to those skilled in the art. Such methods include immunoassays, for example immunohistochemistry (IHC), immunofluorescence (IF), immunoblotting, flow cytometry (e.g., FACS™) or enzyme-linked immunosorbent assay (ELISA), and the like.

In certain embodiments, determining the expression of Ambra-1 in the OPSCC tissue sample comprises:
contacting the OPSCC tissue with a ligand specific for Ambra-1, wherein the presence of Ambra-1 creates an Ambra-1-ligand complex; and
detecting and/or quantifying the Ambra-1-ligand complex.

In some embodiments, the ligand comprises an anti-Ambra-1 antibody or aptamer. In some embodiments, the anti-Ambra-1 antibody or aptamer binds specifically to a protein (or region thereof) comprising SEQ ID NO: 21, 26, 27, 28, 29, 30, 31 and/or 32.

As used herein, the term “aptamer” refers to a non-naturally occurring oligonucleotide that can form a three-dimensional structure that binds to another molecule with high affinity and specificity. As used herein, the terms “nucleic acid ligand” and “aptamer” may be used interchangeably. Aptamer specificity may be comparable or even higher than that of an antibody.

Aptamers have the ability to fold into a variety of complex, sequence-specific tertiary conformations, meaning that they can bind a wide range of targets and rival antibodies in their potential diversity. It is recognized that affinity interactions are a matter of degree; however, in this context, the “specific binding affinity” of an aptamer for its target means that the aptamer binds to its target, i.e. a target molecule, generally with a much higher degree of affinity than it binds to other, non-target, components in a mixture or sample.

An “aptamer” or “nucleic acid ligand” is a set of copies of one type or species of nucleic acid molecule that has a particular nucleotide sequence. An aptamer can include any suitable number of nucleotides. “Aptamers” refers to more than one such set of molecules. Different aptamers can have either the same or different numbers of nucleotides. Aptamers may be DNA or RNA and may be single stranded, double stranded, or contain double stranded regions. In one embodiment, the aptamer comprises single-stranded DNA (ss-DNA) or single stranded RNA (ss-RNA).

In certain embodiments, the ligand specific for Ambra-1 is an antibody as described further below.

The amino acid sequences of human Ambra-1 is provided herein as an example. However, it will be appreciated that variants of these sequences may be known or identified. In some embodiments, the subject is a non-human mammal. It should therefore also be appreciated that references herein to Ambra-1 include the sequences of non-human homologues thereof.

Aptly, the ligand may be used in combination with one or more capture agents. Thus, in some embodiments, the step of detecting and/or quantifying the Ambra-1-ligand complex comprises contacting the OPSCC tissue sample(s) (or the section(s) or portion(s) thereof) with at least one capture agent.

In certain embodiments, the capture agent comprises a binding moiety and a detection moiety. In some embodiments, the binding moiety is a secondary antibody which binds specifically to the ligand. For example, the binding moiety may be a universal anti-IgG antibody that is capable of binding to primary antibodies used as the ligand.

In some embodiments, the method further comprises one or more wash steps to remove unbound ligand and, optionally, unbound capture agents.

In certain embodiments, the expression of Ambra-1 is determined using an antibody (or aptamer) against Ambra-1 as described herein. Typically, the expression of Ambra-1 is determined by contacting the OPSCC tissue with the antibodies (or aptamers) and detecting the presence of the bound antibodies (or aptamers). For example, presence of the bound antibodies (or aptamers) may be detected by visualizing the antibodies (or aptamers) in the OPSCC tissue sample with reagent(s) that generate detectable signal(s) (e.g. detection moieties as described herein).

In some embodiments, the method comprises contacting the OPSCC tissue with the antibody (or aptamer) under conditions permissive for binding of the anti-Ambra-1 antibody (or aptamer) to Ambra-1 and detecting whether a complex is formed between the anti-Ambra-1 antibody (or aptamer) and Ambra-1.

In some embodiments, the ligand comprises a detection moiety (e.g. a fluorescent label). A detection moiety enables the direct or indirect detection and/or quantification of the complexes formed.

In some embodiments, the method further comprises comparing the expression of Ambra-1 with a reference tissue or levels obtained therefrom. For example, the reference may comprise levels of Ambra-1 expression that are characteristic of normal tissue. In some embodiments, reference levels of Ambra-1 are obtained by determining the expression of Ambra-1 in a reference tissue.

In certain embodiments, the expression of Ambra-1 is determined by visual or automated assessment and optionally compared to reference levels. Optionally, the reference levels are an internal reference, e.g. reference levels corresponding to a pre-defined intensity and/or pattern of Ambra-1 expression.

In certain embodiments, the expression of Ambra-1 is determined in the nucleus and/or cytoplasm of cells within the SCC tissue. For example, the % of Ambra-1 positive cells may be scored and optionally compared to reference tissue or levels.

In certain embodiments, the expression of Ambra-1 is scored on the basis of the intensity and/or proportion of positive cells in the OPSCC tissue sample. Scoring methods have been described and are well known to one of ordinary skill in the art.

In certain embodiments, a H-score may be calculated (McCarty et al., Cancer Res. 1986 46(8 Supl):4244s-4248s). An intensity score may be defined as follows:

| | |
|---|---|
| 1= | faint/barely perceptible partial (e.g. weak) staining in the cells |
| 2= | moderate staining of the cells; |
| 3= | strong staining of the cells. |

The H score combines components of staining intensity with the percentage of positive cells. It has a value between 0 and 300 and is defined as:

1*(percentage of cells staining at 1+intensity);+2*(percentage of cells staining at 2+intensity);+3*(percentage of cells staining at 3+intensity);=H score.

A H-score of 140 or above may indicate retained or increased expression of Ambra-1.

Conversely, a H-score of less than 140 may indicate decreased or loss in expression of Ambra-1.

In certain embodiments, “weak”, “moderate” or “strong” staining of the cells is relative to levels of Ambra-1 characteristic of the reference or normal tissue.

In certain embodiments, the method of comparing the expression of Ambra-1 comprises outputting, optionally on a computer, (i) an indication of the expression levels of Ambra-1 and (ii) this indicates whether the subject is suitable or not for de-escalated treatment.

Antibodies Against Ambra-1

In certain embodiments of the invention, antibodies against Ambra-1 are used to determine the expression of Ambra-1 in an OPSCC tissue sample obtained from the subject.

Antibodies against Ambra-1 include any polyclonal antibodies, any monoclonal antibodies, including chimeric antibodies, humanized antibodies, bi-specific antibodies and domains and fragments of monoclonal antibodies including Fab, Fab′, F(ab′)2, scFv, dsFv, ds-scFv, dimers, minibodies, diabodies, and multimers thereof. Monoclonal antibodies can be fragmented using conventional techniques. Monoclonal antibodies may be from any animal origin, including birds and mammals (e.g., human, murine, donkey, sheep, rabbit, goat, guinea pig, camel, horse, or chicken), transgenic animals, or from recombinant sources. Typically, the monoclonal antibodies against Ambra-1 are fully human. Monoclonal antibodies may be prepared using any methods known to those skilled in the art, including by recombination.

Typically, the antibody against Ambra-1 is isolated. An “isolated” antibody is an antibody that has been identified and separated and/or recovered from a component of its natural environment. Contaminant components of its natural environment are materials that would interfere with diagnostic or therapeutic uses for the antibody, and may include enzymes, hormones, and other proteinaceous or non-proteinaceous solutes. In certain embodiments, the antibody will be purified (1) to greater than 95% by weight of antibody as determined by the Lowry method, and most preferably more than 99% by weight, (2) to a degree sufficient to obtain at least 15 residues of N-terminal or internal amino acid sequence by use of a spinning cup sequenator, or (3) to homogeneity by SDS-PAGE under reducing or nonreducing conditions using Coomassie blue or, silver stain.

In certain embodiments, the antibody against Ambra-1 is a fragment that specifically binds Ambra-1.

An “antibody fragment” is a portion of an intact antibody that includes an antigen binding site of the intact antibody and thus retaining the ability to bind Ambra-1. Antibody fragments include:
(i) Fab fragments, having V_{L}, C_{L}, V_{H} and CH1 domains;
(ii) Fab′ fragments, which is a Fab fragment having one or more cysteine residues at the C-terminus of the CH1 domain;
(iii) Fd fragments having V_{H} and CH1 domains;
(iv) Fd′ fragments having V_{H} and CH1 domains and one or more cysteine residues at the C-terminus of the CH1 domain;
(v) Fv fragments having the V_{L} and V_{H} domains of a single arm of an antibody;
(vi) dAb fragments (Ward et al., Nature 341, 544-546 (1989)) which consist of a V_{H} domain;
(vii) isolated CDR regions, including any one or more of SEQ ID Nos 1 to 6;
(viii) F(ab′)2 fragments, a bivalent fragment including two Fab′ fragments linked by a disulphide bridge at the hinge region;
(ix) single chain antibody molecules (e.g. single chain Fv; scFv) (Bird et al, Science 242:423-426 (1988); and Huston et al., PNAS (USA) 85:5879-5883 (1988));
(x) “diabodies” with two antigen binding sites, comprising a heavy chain variable domain (VH) connected to a light chain variable domain (VL) in the same polypeptide chain (see, e.g., EP 404,097 and WO 93/11161;
(xi) “linear antibodies” comprising a pair of tandem Fd segments (VH-CH1-VH-CH1) which, together with complementary light chain polypeptides, form a pair of antigen binding regions (Zapata et al. Protein Eng. 8 (10): 1057-1062 (1995); and U.S. Pat. No. 5,641,870).

Typically, the antibody against Ambra-1 is a recombinant monoclonal antibody. A “recombinant monoclonal antibody” is an antibody or antibody fragment produced using recombinant antibody coding genes. In certain embodiments, the antibodies of the invention are generated from a human combinatorial antibody library (e.g. HuCAL, BioRad).

Typically, the antibody against Ambra-1 is a monovalent Fab or bivalent Fab fragment. A “bivalent Fab fragment” may be considered as equivalent to a F(ab′)2 fragment and formed via dimerization. For example, a bivalent Fab fragment is formed via dimerization of a synthetic double helix loop helix motif (dHLX) or a bacterial alkaline phosphatase (AP) domain. In certain embodiments, the antibody against Ambra-1 comprises a dimerization domain sequence and one or more linker sequences.

In certain embodiments, the antibody against Ambra-1 is a recombinant monoclonal antibody fragment converted into an immunoglobulin (Ig) format. For example, when an Fc region is required, the variable heavy and light chain genes may be cloned into vectors with the desired constant regions and co-transfected for expression in mammalian cells using methods known to those skilled in the art. In certain embodiments, antibody fragments are converted to human IgA, IgE, IgG1, IgG2, IgG3, IgG3 or IgM.

In certain embodiments, the antibody against Ambra-1 is labelled with at least one epitope tag. Typical epitope tags include His6, Flag (e.g. DYKDDDDK), V5 (e.g. GKPIPNPLLGLDST), Strep (e.g. WSHPQFEK) and/or any combination thereof. Typically, the antibody against Ambra-1 is a monovalent Fab or bivalent Fab fragment with one or more (e.g. two) epitopes. In certain embodiments, the antibody against Ambra-1 is conjugated to an enzyme and/or fluorescent label.

In certain embodiments, the antibody specifically binds to Ambra-1. In other words, the antibody against Ambra-1 may bind Ambra-1 with a binding dissociation equilibrium constant (K_{D}) of less than about 30 nM, less than about 20 nM, less than about 10 nm, less than about 1 nm or less than about 200 μm. The skilled person would understand techniques for measuring binding strengths (e.g. koff-rate determination; ‘secondary screening’) include, for example, Bio-Layer Interferometry (e.g. using the Pall ForteBio Octet® System).

In certain embodiments, the antibody against Ambra-1 is available from a commercial supplier. For example, the antibody against Ambra-1 may be AbCAM Ab69501 as described herein. In other embodiments, the antibody against Ambra-1 may be BioRad AbD33473 as described herein.

In certain embodiments, the antibody against Ambra-1 comprises the following heavy chain variable domain complementarity determining regions (CDRs):

(a) HCDR1 comprising the amino acid sequence of SEQ ID NO: 1;

(b) HCDR2 comprising the amino acid sequence of SEQ ID NO: 2; and/or

(c) HCDR3 comprising the amino acid sequence of SEQ ID NO: 3.

In certain embodiments, the antibody against Ambra-1 further comprises the following light chain variable domain CDRs:

(d) LCDR1 comprising the amino acid sequence of SEQ ID NO: 4

(e) LCDR2 comprising the amino acid sequence of SEQ ID NO: 5; and/or

(f) LCDR3 comprising the amino acid sequence of SEQ ID NO: 6.

As used herein, the term “Complementarity Determining Regions” (CDRs) refers to the amino acid residues of an antibody variable domain the presence of which are necessary for antigen binding. Each variable domain typically has three CDR regions identified as CDR1, CDR2 and CDR3. Each complementarity determining region may comprise amino acid residues from a “complementarity determining region” as defined by Kabat (i.e. about residues 24-34 (L1), 50-56 (L2) and 89-97 (L3) in the light chain variable domain and 31-35 (H1), 50-65 (H2) and 95-102 (H3) in the heavy chain variable domain; Kabat et al., Sequences of Proteins of Immunological Interest, 5th Ed. Public Health Service, National Institutes of Health, Bethesda, Md. (1991)) and/or those residues from a “hypervariable loop” (i.e. about residues 26-32 (L1), 50-52 (L2) and 91-96 (L3) in the light chain variable domain and 26-32 (H1), 53-55 (H2) and 96-101 (H3) in the heavy chain variable domain; Chothia and Lesk J. Mol. Biol. 196:901-917 (1987)). In some instances, a CDR region can include amino acids from both a CDR region defined according to Kabat and a hypervariable loop.

Unless otherwise specified herein, numbering of amino acid residues in the Fc region or constant region is according to the EU numbering system, also called the EU index, as described in Kabat et al.

In certain embodiments, the antibody against Ambra-1 further comprises the following heavy chain variable domain framework regions (FRs):

(a) HCFR1 comprising the amino acid sequence of SEQ ID NO:7;

(b) HCFR2 comprising the amino acid sequence of SEQ ID NO:8;

(c) HCFR3 comprising the amino acid sequence of SEQ ID NO: 9; and/or

(d) HCFR4 comprising the amino acid sequence of SEQ ID NO: 10.

In certain embodiments, the antibody against Ambra-1 further comprises the following light chain variable domain FRs:

(e) LCFR1 comprising the amino acid sequence of SEQ ID NO:11;

(f) LCFR2 comprising the amino acid sequence of SEQ ID NO: 12;

(g) LCFR3 comprising the amino acid sequence of SEQ ID NO: 13; and/or

(h) LCFR4 comprising the amino acid sequence of SEQ ID NO: 14.

As used herein, “Framework regions (FRs)” are those variable domain residues other than the CDR residues. Each variable domain typically has four FRs identified as FR1, FR2, FR3 and FR4. If the CDRs are defined according to Kabat, the light chain FR residues are positioned at about residues 1-23 (LCFR1), 35-49 (LCFR2), 57-88 (LCFR3), and 98-107 (LCFR4) and the heavy chain FR residues are positioned about at residues 1-30 (HCFR1), 36-49 (HCFR2), 66-94 (HCFR3), and 103-113 (HCFR4) in the heavy chain residues.

If the CDRs comprise amino acid residues from hypervariable loops, the light chain FR residues are positioned about at residues 1-25 (LCFR1), 33-49 (LCFR2), 53-90 (LCFR3), and 97-107 (LCFR4) in the light chain and the heavy chain FR residues are positioned about at residues 1-25 (HCFR1), 33-52 (HCFR2), 56-95 (HCFR3), and 102-113 (HCFR4) in the heavy chain residues. In some instances, when the CDR comprises amino acids from both a CDR as defined by Kabat and those of a hypervariable loop, the FR residues will be adjusted accordingly. For example, when CDRH1 includes amino acids H26-H35, the heavy chain FR1 residues are at positions 1-25 and the FR2 residues are at positions 36-49.

In certain embodiments, the antibody against Ambra-1 further comprises an antibody variable domain comprising:

(a) a V_{H} sequence having at least 90%, at least 95%, at least 96%, at least 97%, at least 98%, at least 99% or more sequence identity to the amino acid sequence of SEQ ID NO: 15;

(b) a V_{L} sequence having at least 90%, at least 95%, at least 96%, at least 97%, at least 98%, at least 99% or more sequence identity to the amino acid sequence of SEQ ID NO 16; or

(c) a V_{H} sequence as in (a) and a V_{L} sequence as in (b).

In certain embodiments, the antibody against Ambra-1 comprises:

(d) a V_{H} sequence comprising SEQ ID NO: 15;

(e) a V_{L} sequence comprising SEQ ID NO: 16; or

(f) a V_{H} sequence as in (d) and a V_{L} sequence as in (e).

As used herein, “antibody variable domain” refers to the portions of the light and heavy chains of antibody molecules that include amino acid sequences of the CDRs (i.e., CDR1, CDR2, and CDR3) and the FRs (i.e., FR1, FR2, FR3 and FR4). V_{H} refers to the variable domain of the heavy chain. V_{L} refers to the variable domain of the light chain.

As used herein, “sequence identity” refers to a sequence having the specified percentage of amino acid residues that are the same, when compared and aligned (introducing gaps, if necessary) for maximum correspondence, not considering any conservative amino acid substitutions as part of the sequence identity. The percent identity can be measured using sequence comparison software or algorithms or by visual inspection. Various algorithms and software are known in the art that can be used to obtain alignments of amino acid sequences. Suitable programs to determine percent sequence identity include for example the BLAST suite of programs available from the U.S. Government's National Center for Biotechnology Information BLAST web site. Comparisons between two sequences can be carried using either the BLASTN or BLASTP algorithm. BLASTN is used to compare nucleic acid sequences, while BLASTP is used to compare amino acid sequences. ALIGN, ALIGN-2 (Genentech, South San Francisco, Calif.) or MegAlign, available from DNASTAR, are additional publicly available software programs that can be used to align sequences. One skilled in the art can determine appropriate parameters for maximal alignment by alignment software. In certain embodiments, the default parameters of the alignment software are used.

In certain embodiments, the antibody against Ambra-1 comprises a Fab fragment comprising the Fd chain of SEQ ID NO:17; and/or the light chain of SEQ ID NO:18. Typically, such antibodies further comprise one or more dimerization domain sequences, one or more linker sequences and/or one or more epitope tags as described herein.

The invention provides use of antibodies (or aptamers) against Ambra-1 as described herein.

The invention also provides antibodies (or aptamers) that compete for binding to Ambra-1 with antibodies (or aptamers) as described herein. Typically, competition assays are used to identify an antibody (or aptamer) that competes for binding to Ambra-1. In an exemplary competition assay, immobilized Ambra-1 is incubated in a solution comprising a first labelled antibody (or aptamer) that binds to Ambra-1 and a second unlabeled antibody (or aptamer) that is being tested for its ability to compete with the first antibody (or aptamer) for binding to Ambra-1. The second antibody (or aptamer) may be present in a hybridoma supernatant. As a control, immobilized Ambra-1 may be incubated in a solution comprising the first labelled antibody (or aptamer) but not the second unlabeled antibody (or aptamer). After incubation under conditions permissive for binding of the first antibody (or aptamer) to Ambra-1 excess unbound antibody (or aptamer) may be removed, and the amount of label associated with immobilized Ambra-1 measured. If the amount of label associated with immobilized Ambra-1 is substantially reduced in the test sample relative to the control sample, then that indicates that the second antibody (or aptamer) is competing with the first antibody (or aptamer) for binding to Ambra-1. See, e.g., Harlow et al. Antibodies: A Laboratory Manual. Ch. 14 (Cold Spring Harbor Laboratory, Cold Spring Harbor, N.Y., 1988).

In certain embodiments, antibodies (or aptamers) that compete for binding to Ambra-1 bind to the same epitope (e.g., a linear or a conformational epitope) as the antibodies (or aptamers) described herein. Detailed exemplary methods for mapping an epitope to which an antibody binds are provided in Morris “Epitope Mapping Protocols,” in Methods in Molecular Biology Vol. 66 (Humana Press, Totowa, N.J., 1996).

Certain aspects of the present invention further provide isolated nucleic acids that encode any of the antibodies (or aptamers) described herein. Also provided is a vector (e.g., an expression vector) comprising the nucleic acid for expressing any of the antibodies (or aptamers) described herein. Also provided are host cells comprising the preceding nucleic acids and/or vectors.

Certain aspects of the present invention further provide immunoconjugates comprising any of the antibodies (or aptamers) described herein conjugated to one or more capture agents. As described herein, a capture agent typically comprises a binding and/or detection moiety (e.g. an enzyme and/or fluorescent label).

Certain aspects of the present invention further provide antibodies (or aptamers) that specifically bind to the same epitope as the anti-Ambra-1 antibodies (or aptamers) as described herein. In certain embodiments, the invention provides antibodies (or aptamers) that specifically bind to peptides near the carboxyl-terminus of human Ambra-1.

As used herein, the term “epitope” means a protein determinant capable of specific binding to an antibody. Typically, an epitope comprises chemically active surface groupings of molecules such as amino acids or sugar side chains usually having specific three-dimensional structural and charge characteristics. The epitope may comprise amino acid residues directly involved in the binding and optionally additional amino acid residues that are not directly involved in the binding.

As used herein, an antibody (or aptamer) that “specifically” binds to an epitope refers to an antibody (or aptamer) that recognizes the epitope while only having little or no detectable reactivity with other portions of Ambra-1. Such relative specificity can be determined e.g. by competition assays, foot-printing techniques or mass spectrometry techniques as known in the art.

In certain embodiments, the epitope comprises peptide antigenic determinants within single peptide chains of Ambra-1. In certain embodiments, the epitope comprises conformational antigenic determinants comprising one or more contiguous amino acids on a particular chain and/or on spatially contiguous but separate peptide chains. In certain embodiments, the epitope comprises post-translational antigenic determinants comprising molecular structures (e.g. carbohydrate groups) covalently attached to Ambra-1.

The epitope may comprise any suitable number and/or type of amino acids, in any suitable position as defined herein. For example, the epitope may comprise about 3 to about 10 amino acids, typically about 3 to about 8 amino acids, in or more contiguous or non-contiguous locations with respect to the amino acid sequence of Ambra-1 as set forth in SEQ ID NO: 21, 26 or 27.

In certain embodiments, the invention provides antibodies (or aptamers) that bind to Ambra-1, wherein said antibodies (or aptamers) specifically bind to a region comprising 8, 9, 10, 11, 12, 13, 14, 15, 16, 17, 18, 19, 20 or more of the amino acids 1270 to 1298 of human Ambra-1 sequence shown in SEQ ID NO: 21. Typically, such antibodies (or aptamers) specifically bind to a region comprising amino acids 1280 to 1281 of SEQ ID NO:21, amino acids 1294 to 1296 of SEQ ID NO:21 and/or amino acids 1294 to 1297 of SEQ ID NO:21.

In certain embodiments, the invention provides antibodies (or aptamers) that bind to Ambra-1, wherein said antibodies or aptamers specifically bind to a region comprising 8, 9, 10, 11, 12, 13, 14, 15 or more of CGGSSRGDAAGPRGEPRNR (SEQ ID NO: 26). Typically, such antibodies (or aptamers) specifically bind to a region comprising at least EPRN (SEQ ID NO: 31) or at least EPR of Ambra-1.

In certain embodiments, the invention provides antibodies (or aptamers) that bind to Ambra-1, wherein said antibodies or aptamers specifically bind to a region comprising 8, 9, 10, 11, 12, 13, 14, 15 or more of DGGSSRGDAAGPRGEPRNR (SEQ ID NO:28). Typically, such antibodies (or aptamers) specifically bind to a region comprising at least DG, EPRN (SEQ ID NO:31) and/or EPR of Ambra-1.

In certain embodiments, the invention provides antibodies (or aptamers) that bind to Ambra-1, wherein said antibodies (or aptamers) specifically bind to a region comprising HLLDGGSSR (SEQ ID NO: 29) and/or EPRN (SEQ ID NO: 31) of Ambra-1.

In certain embodiments, the invention provides antibodies (or aptamers) that bind to Ambra-1, wherein said antibodies (or aptamers) specifically bind to a region comprising NHLLDGGSSR

(SEQ ID NO: 30) and/or EPRN (SEQ ID NO: 31) of Ambra-1.

In certain embodiments, the invention provides antibodies (or aptamers) that bind to Ambra-1, wherein said antibodies (or aptamers) specifically bind to a region comprising HLLDGGSSR (SEQ ID NO: 29) and/or EPR of Ambra-1.

In certain embodiments, the invention provides antibodies (or aptamers) that bind to Ambra-1, wherein said antibodies or aptamers specifically bind to a region comprising NHLLDGGSSR (SEQ ID NO: 30) and/or EPR of Ambra-1.

Visualization of Ligands

In certain embodiments, the invention provides a method of labelling Ambra-1 in an OPSCC tissue sample as described herein, the method comprising:
(a) contacting the OPSCC tissue with a ligand specific for Ambra-1 as described herein; and
(b) visualizing the ligand against Ambra-1 in the OPSCC tissue with a reagent that generates a detectable signal.

Typically, the ligand specific for Ambra-1 is an antibody (or aptamer) as described herein.

In certain embodiments, a decrease or loss in the expression of Ambra-1 may be determined (e.g. visualized and/or scored) within the OPSCC tissue as described herein. Alternatively, retained or increased expression of Ambra-1 may be determined (e.g. visualized and/or scored) within the OPSCC tissue as described herein. Typically, IHC (e.g. semi-quantitative automated IHC) is used to determine and/or quantify the expression pattern of Ambra-1 in the OPSCC tissue sample as described herein.

Methods of Treatment

In certain embodiments, the invention provides methods of treating a subject suffering from HPV positive OPSCC. Typically, the subject has been determined as being suitable (or not suitable) for de-escalated treatment as described herein.

If there is retained or increased expression of Ambra-1 in an OPSCC tissue sample obtained from the subject, an adjuvant therapy may be administered to the subject. Typically, the adjuvant therapy is chemotherapy (e.g. cisplatin-based chemotherapy) as described herein.

If there is decreased or loss of Ambra-1 expression in an OPSCC tissue sample obtained from the subject, a de-escalated treatment may be administered to the subject. Typically, the de-escalated therapy reduces or avoids chemotherapy (e.g. cisplatin-based chemotherapy).

In certain embodiments, the invention provides a method of treating the subject comprising administering an adjuvant therapy or de-escalated treatment to the subject.

Ideally, a subject identified as being suitable or not for de-escalated treatment is treated as soon as possible. For example, a subject may be treated immediately or shortly after being identified as suitable or not for de-escalated treatment.

For subjects that are not suitable for de-escalated treatment, treatment with an adjuvant therapy may be carried out prior to any primary treatment. Typically, treatment with an adjuvant therapy is carried out during and/or after a primary treatment.

In some embodiments, the de-escalated treatment or adjuvant therapy is administered to the subject no more than 12 weeks, no more than 10 weeks, no more than 6 weeks, no more than 4 weeks, no more than 2 weeks or no more than 1 week after the subject is identified as being suitable for adjuvant therapy or de-escalated treatment.

The de-escalated treatment or adjuvant therapy may be administered in an amount effective to prevent, inhibit or delay the development of OPSCC. Suitable doses and dosage regimes for a given subject and therapeutic agent can be determined using a variety of different methods, such as body surface area or body weight, or in accordance with specialist literature and/or individual hospital or veterinary protocols. Doses may be further adjusted following consideration of a subject's neutrophil count, renal and hepatic function, and history of any previous adverse effects to the therapeutic agent. Doses may also differ depending on whether a therapeutic agent is used alone or in combination.

The skilled person will recognize that further modes of administration, dosages of therapeutic agents and treatment regimens can be determined by the treating physician according to methods known in the art.

Assays

Certain aspects of the present invention provide in vitro assays for determining whether or not a subject suffering from OPSCC is suitable or not for de-escalated treatment.

Typically, the assay comprises contacting an OPSCC tissue sample with a ligand against Ambra-1 as described herein. In the assay, the presence of Ambra-1 creates an Ambra-1-ligand complex. The assay may further comprise detecting and/or quantifying the Ambra-1-ligand.

In some embodiments, the ligand comprises a detection moiety (e.g. a fluorescent label). A detection moiety enables the direct or indirect detection and/or quantification of the complexes formed.

Aptly, the ligand may be used in combination with one or more capture agents. Thus, in some embodiments, the step of detecting and/or quantifying the Ambra-1-ligand complex comprises contacting the SCC tissue sample(s) (or the section(s) or portion(s) thereof) with at least one capture agent. Aptly, a capture agent which binds specifically to the ligand may be used to detect and/or quantify the Ambra-1-ligand complex.

In some embodiments, the capture agent comprises a binding moiety and a detection moiety. In some embodiments, the binding moiety is a secondary antibody which binds specifically to the first and/or second ligands. For example, the binding moiety may be a universal anti-IgG antibody that is capable of binding to primary antibodies used as the first and second ligands.

In some embodiments, the method further comprises one or more wash steps to remove unbound first and second ligands and, optionally, unbound capture agents.

In some embodiments, the in vitro assay comprises:
contacting an OPSCC tissue sample obtained from the subject with a ligand specific for Ambra-1, wherein the presence of Ambra-1 creates an Ambra-1-ligand complex;
washing the OPSCC tissue sample to remove unbound ligands;
contacting the OPSCC tissue sample with a capture agent, wherein the capture agent comprises a detection moiety and a binding moiety specific for the ligand;
washing the OPSCC tissue sample to remove unbound capture agent; and
detecting and/or quantifying the capture agent present in the OPSCC tissue sample.

Aptly, a suitable detection moiety is selected from a fluorescent moiety, a luminescent moiety, a bioluminescent moiety, a radioactive material, a colorimetric moiety, a nanoparticle having suitable detectable properties, a chromogenic moiety, biotin or an enzyme.

Suitable fluorescent moieties include fluorescent proteins (such as phycoerythrin (PE), peridinin-chlorophyll-protein complex (PerCP) and allophycocyanin (APC)) fluorescent dyes (such as Fluorescein Isothiocyanate (FITC), rhodamines (Rs) and cyanines (Cys)), fluorescent tandem complexes (such as Allophycocyanin-Cyanine 7 (APC-Cy7), Peridinin-Chlorophyll-Protein complex-Cyanine 5 (PerCP-cy5) and Phycoerythrin-Texas Red (PE-TexasRed)), and nanocrystals (such as QDot 525, QDot 545 and QDot 625). The presence of Ambra-1-ligand complexes can be detected using fluorescence microscopy via the use of fluorescent ligands or a capture agent comprising a fluorescent detection moiety.

In embodiments where the detection moiety comprises an enzyme, the presence of the Ambra-1-ligand complex can be detected and/or quantified by detecting and/or quantifying the reaction product of a reaction of a substrate catalyzed by the enzyme. In these embodiments, the method further comprises adding a substrate of the enzyme and detecting and/or quantifying the product of the reaction performed on the substrate by the enzyme. For example, the reaction may result in the production of a colored precipitate, which would be detected using light microscopy. Suitable enzymes include, for example, alkaline phosphatase and horseradish peroxidase. A chromogenic substrate of alkaline phosphatase is PNPP (p-Nitrophenyl Phosphate, Disodium Salt). PNPP produces a yellow water-soluble reaction product that absorbs light at 405 nm. Chromogenic substrates of horseradish peroxidase include ABTS (2,2′-Azinobis [3-ethylbenzothiazoline-6-sulfonic acid]-diammonium salt), which yields a green reaction product, OPD (o-phenylenediamine dihydrochloride) which yields a yellow-orange reaction product, and TMB (3,3′,5,5′-tetramethylbenzidine) soluble substrates yield a blue color when detecting HRP. Other suitable enzyme-substrate combinations, methods of detecting the Ambra-1-ligand complex, and suitable detection moieties will be known to those skilled in the art.

In some embodiments, the first and/or second ligand or the capture agent is immobilized on a solid phase surface, for example a microarray, slide, well or bead.

In some embodiments, the expression of Ambra-1 is detected and/or quantified by visual assessment, for example, microscopy. In other embodiments, the expression of Ambra-1 is detected and/or quantified by an automated slide scanner.

In certain embodiments, the method of detecting and/or quantifying the Ambra-1-ligand complex comprises outputting, optionally on a computer, an indication of whether the one or more complexes are present or absent and this indicates whether or not the subject is suitable for adjuvant therapy and/or de-escalated treatment.

Kits

The invention also provides a kit for determining whether or not a subject suffering from OPSCC is suitable for de-escalated treatment. Typically, the kit comprises a ligand against Ambra-1 as described herein.

In certain embodiments, the kit further comprises instructions for using the kit to determine whether or not the subject is suitable for de-escalated treatment.

In certain embodiments, the kit further comprises at least one capture agent. Typically, a capture agent comprises a detection moiety and/or a binding moiety as described herein.

In certain embodiments, the detection moiety is an enzyme (e.g. alkaline phosphatase) and the kit further comprises a substrate of the enzyme.

Typically, the kit may further comprise one or more additional components such as reagents and/or apparatus necessary for carrying out an in vitro assay, e.g. buffers, fixatives, wash solutions, blocking reagents, diluents, chromogens, enzymes, substrates, test tubes, plates, pipettes etc.

The kit of certain embodiments of the invention may advantageously be used for carrying out a method of certain embodiments of the invention and could be employed in a variety of applications, for example in the diagnostic field or as a research tool. It will be appreciated that the parts of the kit may be packaged individually in vials or in combination in containers or multi-container units. Typically, manufacture of the kit follows standard procedures which are known to the person skilled in the art.

### EXAMPLES

### Example 1—Retention of Ambra-1 Expression by Primary OPSCC Identifies High Risk Tumour Subsets

Ambra-1 expression was analysed in a discovery cohort of HPV-positive and negative OPSCC cases identified as part of the UK HPV prevalence study (Schache et al 2016 Cancer Res 76:6598). Consistent with current literature, correlation of HPV status with overall survival (OS) in 91 cases revealed a significant reduction in OS for patients with HPV-negative OPSCC to just 32% compared to 63% for patients with HPV-positive OPSCC (Kaplan Meier, Log rank test, P=0.0012 HR 2.87 (95% CI 1.54-5.35, FIG. 2). Automated immunohistochemical analysis of Ambra-1 expression in triplicate 1 mm tissue microarrays (FIG. 1) derived from 84 cases of HPV positive or negative OPSCC also revealed retention of Ambra-1 expression (h score> or =140) was associated with reduced OS to 28.7% compared to 63% for patients with OPSCC in which Ambra-1 expression was reduced (h score<140, FIG. 3). Furthermore, sub-analysis of Ambra-1 expression in 39 HPV-positive OPSCC cases again revealed high Ambra-1 expression (h score> or = to 140) was associated with reduced OS of only 30.1% versus 74.3% in HPV-positive tumours with low Ambra-1 (h-score<140, FIG. 4). Collectively these data confirm Ambra-1 as a prognostic biomarker independent of HPV status, and importantly as a biomarker with clinical utility in the identification of HPV-positive OPSCC cases at increased risk of death informing decisions on treatment de-escalation.

OPSCC Patient Discovery Tissue Cohort and Immunohistochemical Analysis of AMBRA1

Retrospective OPSCC patient samples were identified as part of the UK HPV Prevalence Study (Schache et al, 2016 Cancer Res 76: 6598-6606). 170 cases of oro-pharyngeal squamous cell carcinomas were identified at Newcastle Hospital NHS Foundation Trust and tissue microarrays (TMA) were constructed from formalin-fixed paraffin embedded tissue blocks. Three 1 mm cores per case were transferred to the TMA recipient block. The HPV status of the samples was determined by p16 immunohistochemistry, high-risk HPV DNA in situ hybridisation and HPV PCR as previously described (Schache et al., 2016). Overall survival data (time from diagnosis to last follow up appointment or death) were available for 91 patients and 84 samples provided sufficient tissue for AMBRA1 immunohistochemistry. Ambra-1 was stained on a Benchmark XT autostainer (Ventana Medical Systems Ltd) using a primary recombinant peptide antibody to human Ambra-1 (AMLo Biosciences Ltd), and images scanned for analysis using an Aperio AT2 slide scanner and e slide manager software (Leica Biosystems Ltd). Staining was scored by a pathologist (MR) and independent investigator (RE) using an H score (Robinson et al, 2019).

A receiver operating characteristic curve (ROC) was built using the continuous classification of AMBRA1 H-score as the discrimination variable for overall survival. The optimum cut-point was identified at an H-score of 140 with a sensitivity of 55% (95% CI 39.83%-69.29%), specificity of 62.79% (95% CI 47.86%-75.62%) and a likelihood ratio of 1.48. Binary scores of 0 for low-risk (H-score<140) and 1 for high-risk (H-score> or = to 140) were applied for further Kaplan-Meier survival curve analysis (Ellis et al., 2019). Other variables assessed included age, sex, HPV status and AMBRA1 tumour positivity. All statistical analysis were undertaken using GraphPad Prism 8 software.

### Example 2—Production and Validation of Anti-Ambra-1 Antibodies

Anti-Ambra-1 antibodies were produced using BioRad HuCAL PLATINUM® antibody generation technology and CysDisplay® technology. HUCAL stands for ‘Human Combinatorial antibody library’, which is a synthetic (generated by de novo gene synthesis) antibody library containing human antibody gene sequences covering more than 95% of the human structural gene repertoire (45 billion antibodies) that are cloned in E. coli phagemid vectors. Each E. coli phage contains one of the 45 billion antibody genes and displays the corresponding antibody on their surface in Fab format, by means of a disulfide linkage between Fab and gene III protein (CysDisplay).

Antigens of Ambra-1 (a synthesized peptide) were used to isolate the antibodies described herein. The antigens were immobilized on to a solid support (i.e., ELISA microtiter plates or covalently coupled to magnetic beads), before the HuCAL library presented on phage was incubated with the antigens. Non-specific antibodies were removed by washing and specific antibody phages eluted by adding a reducing agent.

CysDisplay technology, where the Fab antibody fragment is linked to the phage by a disulphide bond that is easily cleavable rather than a conventional peptide bond, was used to allow more efficient elution of high affinity phages with reducing agents during antibody selection (Bio-Rad). This ensured that high affinity antibodies were not lost during selection, a common problem with more traditional panning phage display methods.

The specific antibody phages were used to infect an E. coli culture along with helper phages, allowing the enriched antibody phage library to be used for subsequent rounds of panning (usually 2-3 rounds of enrichment panning).

After panning, the phagemid DNA encoding the enriched antibody population was isolated as a pool and subcloned into a Fab expression vector containing antibiotic resistance. The vector format chosen was a bivalent Fab (Fab-A-FH) formed with dimerization of bacterial alkaline phosphatase, with two tags Flag (DYKDDDK) and His 6 (His6). E. coli was then transformed with the Fab expression vector ligation mixture and plated on agar plates containing antibiotic. Each growing colony represents a monoclonal antibody and was picked and grown in a 384 well microtiter plate. Antibody expression was induced and the culture harvested and lysed to release the antibodies.

Culture lysates were screened primarily for specific antigen binding to antigens by indirect ELISA. 95 ELISA-positive antibody clones, derived from the primary screening, were ranked according to their binding strength (koff-rate determination; ‘secondary screening’) as measured by Bio-Layer Interferometry using the Pall ForteBio Octet® System. Antibodies were then selected according to both antigen specificity and binding strength. Hits from the primary or secondary antibody screening procedures were sequenced to identify unique antibodies. The Fab antibodies with unique sequences were expressed in E. coli and purified using one-step affinity chromatography. Purified antibodies were tested by QC ELISA for required specificity. This QC ELISA screen was performed on native as well as denatured antigen due to the final antibody application of immunohistochemistry, where antigens during the tissue processing may be denatured, as well as immobilized control proteins Glutathione S-transferase, BSA (carrier protein), N1-CD33-His6 (the ectodomain of human CD33 fused to the N1 domain of the g3p filamentous phage M13) used for calculation of background. Purity was assessed by Coomassie® staining of a sodium dodecyl sulfate-polyacrylamide gel (SDS-PAGE) and concentration measured by UV absorbance at 280 nm.

13 Ambra-1 recombinant monoclonal antibody Fab fragments were obtained and used for subsequent validation.

The recombinant human HuCAL antibody fragment antibodies were validated in the first instance using immunohistochemistry protocols on normal skin tissue (where Ambra-1 is expressed) and in a selection of AJCC stage I melanoma tumor tissue (where expression of Ambra-1 is maintained in the epidermis overlying the tumor or lost). In all instances the staining of the HuCAL antibodies were compared to commercially available Ambra-1 antibodies by Abcam in the same tissue. Negative control of omitting the primary antibody and using anti-Flag (HuCAL antibodies) or anti-Rabbit (Abcam antibodies) secondary antibodies was also included in each instance.

### Example 3—Epitope Mapping of Anti-Ambra-1 Antibodies

Epitopes of Ambra-1 recognized by anti-Ambra-1 antibodies were mapped using linear, conformational and replacement analysis epitope mapping (Pepscan Presto BV) using established techniques (Timmerman et al (2007). J. Mol. Recognit. 20: 283-299; Langedijk et al. (2011) Analytical Biochemistry 417: 149-155).

The anti-Ambra-1 antibody was tested on arrays with overlapping linear peptides and looped peptides, based on the C-terminal sequence of Ambra-1:

VSLPSAEGPTLHCELTNNNHLLDGGSSRGDAAGPRGEPRNR (SEQ ID NO: 27)

The core epitope, based on overlapping peptides in the linear and looped arrays, was determined to be sequence ₃₇ EPR₃₉. A second binding site is apparent only in specific length peptides, and more pronounced in the looped peptide array. Binding to these residues may require a very specific conformation, which in the LOOP11 peptide mimics is provided readily, and can only be induced with the specific residue content in the LIN11 peptides. If so, this suggests that a secondary structure may be formed for recognition of these residues. This recognition occurs only in this length peptide. Thus, it is possible that specific residues need to be aligned precisely to be recognized. This may represent a structure such as a beta turn, in which some residues on opposite strands are required for the formation, allowing the proper positioning of the two identified residues in the loop tip. The location of the two residues in the center of the 11-mers also corroborates such a possibility. In conclusion, the main residues for binding are ₃₇ EPR₃₉, which may be aided by ₂₃DG₂₄ in a specific conformation.

Details of the epitope information is summarized in the Table below. Core binding sites are listed based on overlap of peptides. Underlined sequence highlights the key residues deducted from the replacement analysis (REPNET):

| Anti-Ambra-1 | Epitope |
|---|---|
| REPNET | ₂₂DGGSSRGDAAGPRGEPRNR₄₁ (SEQ ID NO: 28) |
| LIN | ₃₇EPR₃₉ |
| | ₁₉HLLDGGSSR₂₈(SEQ ID NO: 29) |
| LOOP | ₃₇EPR39 |
| | ₁₉NHLLDGGSSR₂₈(SEQ ID NO: 30) |

The reader's attention is directed to all papers and documents which are filed concurrently with or before this specification in connection with this application and which are open to public inspection with this specification, and the contents of all such papers and documents are incorporated herein by reference.

1. A method of determining whether or not a subject suffering from HPV positive oropharyngeal squamous cell carcinoma (OPSCC) is suitable for de-escalated treatment, the method comprising determining the expression of Ambra-1 in an OPSCC tissue sample obtained from the subject, wherein:
(i) retained or increased expression of Ambra-1 indicates the subject is not suitable for de-escalated treatment; or
(ii) decreased or loss in expression of Ambra-1 indicates the subject is suitable for de-escalated treatment.

2. The method of claim 1, wherein the subject has undertaken or is undergoing a primary treatment, wherein the primary treatment is surgery and/or radiation treatment.

3. (canceled)

4. The method of claim 1, wherein the de-escalated treatment avoids adjuvant therapy, wherein the avoided adjuvant therapy is chemotherapy, preferably cisplatin based chemotherapy.

5. (canceled)

6. The method of claim 1, wherein the de-escalated treatment comprises administration of a reduced dosage of radiotherapy and/or chemotherapy as compared to a normal recognised care pathway, wherein the normal recognised care pathway comprises intensity-modulated radiation therapy (IMRT) up to a dosage of about 70Gy plus cisplatin-based chemotherapy up to about 300 mg/m².

7. (canceled)

8. The method of claim 1, wherein the de-escalated treatment comprises administering a therapeutic antibody to the subject, wherein the therapeutic antibody is an epidermal growth factor receptor (EGFR) inhibitor, wherein the EGFR inhibitor is cetuximab.

9. (canceled)

10. The method of claim 1, wherein determining the expression of Ambra-1 in the tissue sample comprises contacting the tissue sample with a ligand specific for Ambra-1, wherein the ligand specifically binds to the region EPRN, HLLDGGSSR and/or NHLLDGGSSR of human Ambra-1 (SEQ ID NO: 21) and/or wherein the ligand specific for Ambra-1 is an antibody or aptamer and/or wherein the method further comprises visualising and/or quantifying the ligand in the tissue sample with a reagent that generates a detectable signal.

11. (canceled)

12. (canceled)

13. (canceled)

14. The method of claim 1, wherein the expression of Ambra-1 is scored based on the intensity and/or percentage of Ambra-1 positive cells in the tissue.

15. The method of claim 14, wherein the intensity and percentage of Ambra-1 positive cells is determined in a fixed area of tissue to obtain a histo (H)-score using the following formula:
1×(% weakly stained cells)+2×(% moderately stained cells)+3×% strongly stained cells);
wherein the H-score is within a range of 0 to 300 and a H-score of 140 or above indicates retained or increased expression of Ambra-1 or a H-score of less than 140 indicates decreased or loss in expression of Ambra-1.

16. A method of treating a subject suffering from HPV positive OPSCC comprising:
(i) determining whether or not the subject is suitable for de-escalated treatment according to the method of claim 1; and
(ii) if the subject is not suitable for de-escalated treatment, administering an adjuvant therapy to the subject.

17. A method of treating a subject suffering from HPV positive OPSCC identified as not suitable for de-escalated treatment according to the method of claim 1, comprising administering an adjuvant therapy to the subject.

18. The method of claim 16, wherein the adjuvant therapy that is administered is chemotherapy, preferably cisplatin-based chemotherapy.

19. A method of treating a subject suffering from HPV positive OPSCC comprising:
(i) determining whether or not the subject is suitable for de-escalated treatment according to the method of claim 1; and
(ii) if the subject is suitable for de-escalated treatment, administering a de-escalated treatment to the subject.

20. A method of treating a subject suffering from HPV positive OPSCC identified as suitable for de-escalated treatment according to the method of claim 1, comprising administering a de-escalated treatment to the subject.

21. The method of claim 19, wherein the de-escalated treatment:
(a) avoids adjuvant therapy, wherein the avoided adjuvant therapy is chemotherapy or cisplatin-based chemotherapy;
(b) comprises administration of a reduced dosage of radiotherapy and/or chemotherapy as compared to a normal recognised care pathway, wherein the normal recognised care pathway comprises intensity-modulated radiation therapy (IMRT) up to a dosage of about 70Gy plus cisplatin-based chemotherapy up to about 300 mg/m²; or
(c) comprises administration of a therapeutic antibody, wherein the therapeutic antibody is an EGFR inhibitor, wherein the EGFR inhibitor is cetuximab.

22. An in vitro assay for determining whether or not a subject suffering from HPV positive OPSCC is suitable for de-escalated treatment, the assay comprising:
contacting OPSCC tissue in a sample obtained from the subject with a ligand specific for Ambra-1, wherein the presence of Ambra-1 creates an Ambra-1-ligand complex; and
detecting and/or quantifying the Ambra-1-ligand complex.

23. The in vitro assay of claim 22, wherein:
(a) the ligand specifically binds to the region EPRN, HLLDGGSSR and/or NHLLDGGSSR of human Ambra-1 (SEQ ID NO: 21);
(b) the ligand specific for Ambra-1 is an antibody or aptamer; and/or
(c) the Ambra-1-ligand complex is detected and/or quantified by visual assessment or by an automated slide scanner.

24. A kit for determining whether or not a subject suffering from HPV positive OPSCC is suitable for de-escalated treatment, the kit comprising a ligand specific for Ambra-1.

25. The kit according to claim 24, further comprising at least one capture agent, wherein the at least one capture agent comprises a detection moiety and/or a binding moiety specific for the ligand specific for Ambra-1.


SEQUENCE LISTING (31 sequences)

SEQ ID NO: 1 - protein, 5 aa, Homo sapiens
SYWIH

SEQ ID NO: 2 - protein, 17 aa, Homo sapiens
TIFPSRSYTTYSPSFQG

SEQ ID NO: 3 - protein, 13 aa, Homo sapiens
DTPSTALKSPFDY

SEQ ID NO: 4 - protein, 13 aa, Homo sapiens
SGSSSNIGYNYVY

SEQ ID NO: 5 - protein, 7 aa, Homo sapiens
ENNKRPS

SEQ ID NO: 6 - protein, 11 aa, Homo sapiens
SSWDSHSNSYV

SEQ ID NO: 7 - protein, 30 aa, Homo sapiens
EVQLVQSGAEVKKPGESLKISCKGSGYSFS

SEQ ID NO: 8 - protein, 14 aa, Homo sapiens
WVRQMPGKGLEWMG

SEQ ID NO: 9 - protein, 32 aa, Homo sapiens
QVTISADKSISTAYLQWSSLKASDTAMYYCAR

SEQ ID NO: 10 - protein, 11 aa, Homo sapiens
WGQGTLVTVSS

SEQ ID NO: 11 - protein, 22 aa, Homo sapiens
DIVLTQPPSVSGAPGQRVTISC

SEQ ID NO: 12 - protein, 15 aa, Homo sapiens
WYQQLPGTAPKLLIY

SEQ ID NO: 13 - protein, 32 aa, Homo sapiens
GVPDRFSGSKSGTSASLAITGLQAEDEADYYC

SEQ ID NO: 14 - protein, 12 aa, Homo sapiens
FGGGTKLTVLGQ

SEQ ID NO: 15 - protein, 122 aa, Homo sapiens
EVQLVQSGAEVKKPGESLKISCKGSGYSFSSYWIHWVRQMPGKGLEWMGTIFPSRSYTTYSPSFQGQVTISADKSISTAYLQWSSLKASDTAMYYCARDTPSTALKSPFDYWGQGTLVTVSS

SEQ ID NO: 16 - protein, 112 aa, Homo sapiens
DIVLTQPPSVSGAPGQRVTISCSGSSSNIGYNYVYWYQQLPGTAPKLLIYENNKRPSGVPDRFSGSKSGTSASLAITGLQAEDEADYYCSSWDSHSNSYVFGGGTKLTVLGQ

SEQ ID NO: 17 - protein, 224 aa, Homo sapiens
EVQLVQSGAEVKKPGESLKISCKGSGYSFSSYWIHWVRQMPGKGLEWMGTIFPSRSYTTYSPSFQGQVTISADKSISTAYLQWSSLKASDTAMYYCARDTPSTALKSPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKS

SEQ ID NO: 18 - protein, 215 aa, Homo sapiens
DIVLTQPPSVSGAPGQRVTISCSGSSSNIGYNYVYWYQQLPGTAPKLLIYENNKRPSGVPDRFSGSKSGTSASLAITGLQAEDEADYYCSSWDSHSNSYVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTEA

SEQ ID NO: 19 - DNA, 2085 bp, Artificial Sequence
  A synthetic sequence encoding the Fd chain of Fab region and tags AP dimerization domain sequence, FLAG tag (DYKDDDDK and His6 tag of the anti-Ambra-1 antibody
gaagtgcaattggtgcagagcggtgcggaagtgaaaaaaccgggcgaaagcctgaaaattagctgcaaaggctccggatatagcttctcttcttactggatccattgggtgcgccagatgccgggcaaaggtctcgagtggatgggcactatcttcccgtctcgtagctacaccacttatagcccgagctttcagggccaggtgaccattagcgcggataaaagcatcagcaccgcgtatctgcaatggagcagcctgaaagcgagcgataccgcgatgtattattgcgcgcgtgacactccgtctactgctctgaaatctccgttcgattactggggccaaggcaccctggtgactgttagctcagcgtcgaccaaaggcccgagcgtgtttccgctggccccgagcagcaaaagcaccagcggcggcaccgccgcactgggctgcctggtgaaagattatttcccggaaccagtgaccgtgagctggaacagcggtgccctgaccagcggcgtgcatacctttccggcggtgctgcaaagcagcggcctgtatagcctgagcagcgttgtgaccgtgccgagcagcagcctgggcacccagacctatatttgcaacgtcaaccataaaccgagcaacaccaaagtcgataaaaaagtcgaaccgaaaagcgaattcaaggctgaaatgcctgttctggaaaaccgggctgctcagggcgatattactacacccggcggtgctcgccgtttaacgggtgatcagactgccgctctgcgtgattctcttagcgataaacctgcaaaaaatattattttgctgattggcgatgggatgggggactcggaaattactgccgcacgtaattatgccgaaggtgcgggcggcttttttaaaggtatagatgccttaccgcttaccgggcaatacactcactatgcgctgaatagaaaaaccggcaaaccggactacgtcaccagctcggctgcatcagcaaccgcctggtcaaccggtgtcaaaacctataacggcgcgctgggcgtcgatattcacgaaaaagatcacccaacgattctggaaatggcaaaagccgcaggtctggcgaccggtaacgtttctaccgcagagttgcaggatgccacgcccgctgcgctggtggcacatgtgacctcgcgcaaatgctacggtccgagcgcgaccagtgaaaaatgtccgggtaacgctctggaaaaaggcggaaaaggatcgattaccgaacagctgcttaacgctcgtgccgacgttacgcttggcggcggcgcaaaaacctttgctgaaacggcaaccgctggtgaatggcagggaaaaacgctgcgtgaacaggcacaggcgcgtggttatcagttggtgagcgatgctgcctcactgaactcggtgacggaagcgaatcagcaaaaacccctgcttggcctgtttgctgacggcaatatgccagtgcgctggctaggaccgaaagcaacgtaccatggcaatatcgataagcccgcagtcacctgtacgccaaatccgcaacgtaatgacagtgtaccaaccctggcgcagatgaccgacaaagccattgaattgttgagtaaaaatgagaaaggctttttcctgcaagttgaaggtgcgtcaatcgataaacaggatcatgctgcgaatccttgtgggcaaattggcgagacggtcgatctcgatgaagccgtacaacgggcgctggagttcgctaaaaaggagggtaacacgctggtcatagtcaccgctgatcacgcccacgccagccagattgttgcgccggataccaaagctccgggcctcacccaggcgctaaataccaaagatggcgcagtgatggtgatgagttacgggaactccgaagaggattcacaagaacataccggcagtcagttgcgtattgcggcgtatggcccgcatgccgccaatgttgttggactgaccgaccagaccgatctcttctacaccatgaaagccgctctggggctgaaaggcgcgccggactataaagatgacgatgacaaaggcgcgccgcaccatcatcaccatcac

SEQ ID NO: 20 - DNA, 645 bp, Homo sapiens
gatatcgtgctgacccagccgccgagcgtgagcggtgcaccgggccagcgcgtgaccattagctgtagcggcagcagcagcaacattggttacaactacgtgtactggtaccagcagctgccgggcacggcgccgaaactgctgatctacgaaaacaacaaacgcccgagcggcgtgccggatcgctttagcggatccaaaagcggcaccagcgccagcctggcgattaccggcctgcaagcagaagacgaagcggattattactgctcttcttgggactctcattctaactcttacgtgtttggcggcggcacgaagttaaccgttcttggccagccgaaagccgccccaagcgtgaccctgtttccgccgagcagcgaagaactgcaagccaacaaagccaccctggtttgcctgatcagcgatttttatccgggtgccgtgaccgtggcctggaaagccgatagcagcccggtgaaagccggcgtggaaaccaccaccccgagcaaacagagcaacaacaaatatgccgccagcagctatctgagcctgaccccggaacagtggaaaagccatcgcagctatagttgtcaagtgacccatgaaggcagcaccgtggaaaaaaccgtggccccgaccgaggcc

SEQ ID NO: 21 - protein, 1298 aa, Homo sapiens
MKVVPEKNAVRILWGRERGARAMGAQRLLQELVEDKTRWMKWEGKRVELPDSPRSTFLLAFSPDRTLLASTHVNHNIYITEVKTGKCVHSLIGHRRTPWCVTFHPTISGLIASGCLDGEVRIWDLHGGSESWFTDSNNAIASLAFHPTAQLLLIATANEIHFWDWSRREPFAVVKTASEMERVRLVRFDPLGHYLLTAIVNPSNQQGDDEPEIPIDGTELSHYRQRALLQSQPVRRTPLLHNFLHMLSSRSSGIQVGEQSTVQDSATPSPPPPPPQPSTERPRTSAYIRLRQRVSYPTAECCQHLGILCLCSRCSGTRVPSLLPHQDSVPPASARATTPSFSFVQTEPFHPPEQASSTQQDQGLLNRPSAFSTVQSSTAGNTLRNLSLGPTRRSLGGPLSSHPSRYHREIAPGLTGSEWTRTVLSLNSRSEAESMPPPRTSASSVSLLSVLRQQEGGSQASVYTSATEGRGFPASGLATESDGGNGSSQNNSGSIRHELQCDLRRFFLEYDRLQELDQSLSGEAPQTQQAQEMLNNNIESERPGPSHQPTPHSSENNSNLSRGHLNRCRACHNLLTFNNDTLRWERTTPNYSSGEASSSWQVPSSFESVPSSGSQLPPLERTEGQTPSSSRLELSSSASPQEERTVGVAFNQETGHWERIYTQSSRSGTVSQEALHQDMPEESSEEDSLRRRLLESSLISLSRYDGAGSREHPIYPDPARLSPAAYYAQRMIQYLSRRDSIRQRSMRYQQNRLRSSTSSSSSDNQGPSVEGTDLEFEDFEDNGDRSRHRAPRNARMSAPSLGRFVPRRFLLPEYLPYAGIFHERGQPGLATHSSVNRVLAGAVIGDGQSAVASNIANTTYRLQWWDFTKFDLPEISNASVNVLVQNCKIYNDASCDISADGQLLAAFIPSSQRGFPDEGILAVYSLAPHNLGEMLYTKRFGPNAISVSLSPMGRYVMVGLASRRILLHPSTEHMVAQVFRLQQAHGGETSMRRVFNVLYPMPADQRRHVSINSARWLPEPGLGLAYGTNKGDLVICRPEALNSGVEYYWDQLNETVFTVHSNSRSSERPGTSRATWRTDRDMGLMNAIGLQPRNPATSVTSQGTQTLALQLQNAETQTEREVPEPGTAASGPGEGEGSEYGASGEDALSRIQRLMAEGGMTAVVQREQSTTMASMGGFGNNIIVSHRIHRSSQTGTEPGAAHTSSPQPSTSRGLLPEAGQLAERGLSPRTASWDQPGTPGREPTQPTLPSSSPVPIPVSLPSAEGPTLHCELTNNNHLLDGGSSRGDAAGPRGEPRNR

SEQ ID NO: 22 - protein, 8 aa, Artificial Sequence
  Synthetic exemplary epitope tag amino acid sequence
DYKDDDDK

SEQ ID NO: 23 - protein, 14 aa, Artificial Sequence
  Synthetic exemplary epitope tag amino acid sequence
GKPIPNPLLGLDST

SEQ ID NO: 24 - protein, 8 aa, Artificial Sequence
  Synthetic exemplary epitope tag amino acid sequence
WSHPQFEK

SEQ ID NO: 25 - protein, 6 aa, Artificial Sequence
  A synthetic His6 tag amino acid sequence
HHHHHH

SEQ ID NO: 26 - protein, 19 aa, Homo sapiens
CGGSSRGDAAGPRGEPRNR

SEQ ID NO: 27 - protein, 41 aa, Homo sapiens
VSLPSAEGPTLHCELTNNNHLLDGGSSRGDAAGPRGEPRNR

SEQ ID NO: 28 - protein, 19 aa, Homo sapiens
DGGSSRGDAAGPRGEPRNR

SEQ ID NO: 29 - protein, 9 aa, Homo sapiens
HLLDGGSSR

SEQ ID NO: 30 - protein, 10 aa, Homo sapiens
NHLLDGGSSR

SEQ ID NO: 31 - protein, 4 aa, Homo sapiens
EPRN